(19) United States
(12) Patent Application Publication
(10) Pub. No.: US 2026/0215794 A1
(43) Pub. Date: Jul. 30, 2026

(54) DEVICES FOR ANATOMICAL OBSTRUCTION EXTRACTION

(71) Applicant: Boston Scientific Medical Device Limited, Galway (IE)

(72) Inventors: Deepak Kumar Sharma, Gurgaon (IN); Sharath Kumar G, Kanakapura (IN)

(73) Assignee: Boston Scientific Medical Device Limited, Galway (IE)

(21) Appl. No.: 19/460,828

(22) Filed: Jan. 27, 2026

Related U.S. Application Data
(60) Provisional application No. 63/750,580, filed on Jan. 28, 2025.

(51) Int. Cl. A61B 17/221

(52) U.S. Cl. CPC A61B 17/221; A61B 2017/2212

(57) ABSTRACT

A medical device for removing stones from a body lumen includes an elongate shaft with inner and outer loops that extend from the shaft. The inner and outer loops are movable between a capture configuration, where the inner loop plane is substantially orthogonal to the outer loop plane, and a release configuration, where the loops are substantially parallel. The elongate shaft includes an outer tubular member, an inner tube within the outer tubular member, and a core wire within the inner tube. The inner loop connects to the core wire while the outer loop connects to the inner tube. A handle secured to the elongate shaft includes a rotatable knob coupled to the core wire and a sliding element that enables translation of the core wire to control loop movement and configuration.

### CROSS-REFERENCE TO RELATED APPLICATIONS

This application claims the benefit of U.S. Provisional Patent Application Serial No. 63/750,580 filed on January 28, 2025, the disclosure of which is incorporated herein by reference.

### TECHNICAL FIELD

The present disclosure is directed to medical devices. More particularly, the present disclosure relates to anatomical obstruction extraction devices that may be utilized for treatment within various anatomical systems and/or subsystems of a subject, including but not limited to the gastrointestinal (GI) tract, the biliary tract, the vasculature, and/or the like.

### BACKGROUND

Anatomical obstructions such as biliary stones, kidney stones, calculi, thrombi, emboli and the like present challenging difficulties for effective and safe removal from body lumens. In some examples, a physician and/or practitioner may find an obstruction within one or more various anatomical systems within a subject that may be greater than 15 mm in its greatest dimension (i.e., width, length, diameter, height, etc.) Obstructions of this magnitude present inherent complications of removal given their size, and the stress that these large obstructions may place on the interior luminal walls of the subject.

Additional complications may arise during the attempted removal of an obstruction as described above. In many procedures, a device bearing a basket may be inserted proximate and/or across and/or along a desired area of treatment (e.g., biliary tract, gastrointestinal tract, blood vessel, or the like). In some cases, depending on the size of the obstruction being removed, the combination of obstruction and basket may become too large to remove, thereby requiring a surgical intervention.

This disclosure provides design, material, method, and use alternatives for medical devices, including but not limited to anatomical obstruction extraction devices. An example may be found in a medical device adapted for removing stones from a body lumen that may include an elongate shaft, an inner loop extendable from the elongate shaft within an inner loop plane, and an outer loop extendable from the elongate shaft within an outer loop plane, wherein the inner loop and/or the outer loop may be movable between a capture configuration in which the inner loop plane is transverse to the outer loop plane and a release configuration in which the inner loop plane is at least substantially parallel with the outer loop plane.

Alternatively or additionally to any of the examples above, the inner loop may be adapted to be rotatable relative to the outer loop.

Alternatively or additionally to any of the examples above, the elongate shaft may be adapted to allow the inner loop to translate relative to the elongate shaft.

Alternatively or additionally to any of the examples above, the elongate shaft may include an outer tubular member, an inner tube extending within the outer tubular member, and a core wire extending within the inner tube.

Alternatively or additionally to any of the examples above, the inner loop may be connected to the core wire and the outer loop may be connected to the inner tube.

Alternatively or additionally to any of the examples above, the medical device may further include a handle secured to the elongate shaft.

Alternatively or additionally to any of the examples above, the handle may include a handle body, a knob rotatably secured relative to the handle body, the knob operably coupled to the core wire such that rotation of the knob causes rotation of the core wire, and a finger ring slidingly secured relative to the handle body, the finger knob operably coupled to the core wire such that sliding the finger ring relative to the handle body causes translation of the core wire.

Alternatively or additionally to any of the examples above, withdrawing the core wire proximally relative to the inner tube may cause the inner loop to collapse.

Alternatively or additionally to any of the examples above, advancing the outer tubular member distally relative to the inner tube may cause the outer tubular member to collapse the outer loop.

Alternatively or additionally to any of the examples above, the inner loop and the outer loop may be made of nitinol.

Alternatively or additionally to any of the examples above, the handle may be adapted to allow a user to translate the inner loop relative to the elongate shaft and the handle may be adapted to allow a user to rotate the inner loop relative to the outer loop.

Alternatively or additionally to any of the examples above, the medical device may further include an outer tube adapted to slidingly fit over the inner tube.

Alternatively or additionally to any of the examples above, the outer tube may be adapted to be pushed distally in order to close the outer loop.

Alternatively or additionally to any of the examples above, in the capture configuration, the inner loop may be at least substantially orthogonal to the outer loop, and in the release configuration, the inner loop may be at least substantially coplanar with the outer loop.

Alternatively or additionally to any of the examples above, the elongate shaft may be adapted to reversibly collapse the outer loop.

Another example may be found in a medical device adapted for removing stones from a body lumen that may include an elongate shaft, an inner loop extendable from the elongate shaft within an inner loop plane, and an outer loop extendable from the elongate shaft within an outer loop plane, wherein the inner loop and/or the outer loop may be movable between a capture configuration in which the inner loop plane is transverse to the outer loop plane and a release configuration in which the inner loop plane is at least substantially parallel with the outer loop plane.

Alternatively or additionally to any of the examples above, the inner loop may be adapted to be rotatable relative to the outer loop.

Alternatively or additionally to any of the examples above, the elongate shaft may be adapted to allow the inner loop to translate relative to the elongate shaft.

Alternatively or additionally to any of the examples above, the elongate shaft may be adapted to reversibly collapse the outer loop.

Alternatively or additionally to any of the examples above, the inner loop and the outer loop may be made of nitinol.

Alternatively or additionally to any of the examples above, the medical device may further include a handle secured to the elongate shaft.

Alternatively or additionally to any of the examples above, the handle may be adapted to allow a user to translate the inner loop relative to the elongate shaft.

Alternatively or additionally to any of the examples above, the handle may be adapted to allow a user to rotate the inner loop relative to the outer loop.

Alternatively or additionally to any of the examples above, the elongate shaft may include an outer tubular member, an inner tube extending within the outer tubular member, and a core wire extending within the inner tube.

Alternatively or additionally to any of the examples above, the inner loop may be connected to the core wire and the outer loop may be connected to the inner tube.

Another example may be found in a medical device adapted for removing stones from a body lumen that may include an elongate shaft including an outer tubular member, an inner tube extending within the outer tubular member, and a core wire extending within the inner tube, an inner loop secured to the core wire, and an outer loop secured to the inner tube, wherein the inner loop and/or the outer loop may be movable between a capture configuration and a release configuration.

Alternatively or additionally to any of the examples above, in the capture configuration, the inner loop may be at least substantially orthogonal to the outer loop, and in the release configuration, the inner loop may be at least substantially coplanar with the outer loop.

Alternatively or additionally to any of the examples above, the inner loop may be adapted to be rotatable between the capture configuration and the release configuration.

Alternatively or additionally to any of the examples above, withdrawing the core wire proximally relative to the inner tube may cause the inner loop to collapse.

Alternatively or additionally to any of the examples above, advancing the outer tubular member distally relative to the inner tube may cause the outer tubular member to collapse the outer loop.

Alternatively or additionally to any of the examples above, the medical device may further include a handle secured to the elongate shaft, the handle including a handle body, a knob rotatably secured relative to the handle body, the knob operably coupled to the core wire such that rotation of the knob causes rotation of the core wire, and a finger ring slidingly secured relative to the handle body, the finger knob operably coupled to the core wire such that sliding the finger ring relative to the handle body causes translation of the core wire.

Another example may be found in a medical device adapted for removing stones from a body lumen that may include a handle including a handle body, a knob rotatably secured relative to the handle body, and a sliding element slidingly secured relative to the handle body, an inner tube extending distally from the handle, a core wire extending distally from the core wire within the inner tube, an inner loop extending distally from the core wire, and an outer loop extending distally from the inner tube, wherein the inner loop and/or the outer loop may be movable between a capture configuration and a release configuration.

Alternatively or additionally to any of the examples above, in the capture configuration, the inner loop may be at least substantially orthogonal to the outer loop, and in the release configuration, the inner loop may be at least substantially coplanar with the outer loop.

Alternatively or additionally to any of the examples above, the medical device may further include an outer tube adapted to slidingly fit over the inner tube.

Alternatively or additionally to any of the examples above, the outer tube may be adapted to be pushed distally in order to close the outer loop.

### BRIEF DESCRIPTION OF THE DRAWINGS

The drawings illustrate the design and utility of configurations of the present disclosure. It should be noted that the figures are not drawn to scale and that elements of similar structures or functions are represented by like reference numerals throughout the figures. The drawings are not to be considered as limiting the scope of the disclosure. The present disclosure is described and explained with additional specificity and detail through the use of the accompanying drawings, in which:

FIG. 1 illustrates a schematic view of an example of accessing the biliary duct of a subject with a medical device system;

FIG. 2 illustrates a schematic view of an obstruction lodged within the biliary duct of a subject;

FIG. 3 illustrates a schematic view of an example medical device that may be used for capturing and retrieving an obstruction from within the biliary duct of a subject;

FIG. 4 illustrates a schematic view of a portion of the example medical device of FIG. 3;

FIG. 5 illustrates a schematic view of a portion of the example medical device of FIG. 3, shown with an inner loop partially collapsed;

FIG. 6 illustrates a schematic view of a portion of the example medical device of FIG. 3, shown with the inner loop and an outer loop both partially collapsed;

FIG. 7 is a cross-sectional view taken along the line 7-7 of FIG. 3;

FIG. 8 illustrates a schematic view of a portion of the example medical device of FIG. 3, shown with the inner and outer loops in a capture configuration forming a basket with an obstruction disposed within the basket;

FIG. 9 illustrates a schematic view of a portion of the example medical device of FIG. 3, shown with the inner and outer loops in a release configuration no longer forming a basket, with the obstruction free to migrate relative to the inner and outer loops; and

FIG. 10 illustrates a schematic view of a portion of the example medical device of FIG. 3, shows with the inner and outer loops in the release configuration with the inner and outer loops free of the obstruction.

While the disclosure is amenable to various modifications and alternative forms, specifics thereof have been shown by way of example in the drawings and will be described in detail. It should be understood, however, that the intention is not to limit the disclosure to the particular configurations described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “a configuration”, “some configurations”, “other configurations”, etc., indicate that the configuration described may include one or more particular features, structures, and/or characteristics. However, such recitations do not necessarily mean that all configurations include the particular features, structures, and/or characteristics. Additionally, when particular features, structures, and/or characteristics are described in connection with one configuration, it should be understood that such features, structures, and/or characteristics may also be used in connection with other configurations whether or not explicitly described unless clearly stated to the contrary.

The following detailed description should be read with reference to the drawings in which similar elements in different drawings are numbered the same. The drawings, which are not necessarily to scale, depict illustrative configurations and are not intended to limit the scope of the disclosure.

The presence of an anatomical obstruction in and/or along the biliary tract can lead to serious conditions, such as, but not limited to, obstructive jaundice, acute suppurative cholangitis, sepsis, and/or gallstone pancreatitis. Prior to the implementation of endoscopic retrograde cholangiopancreatography (ERCP), extraction of an anatomical obstruction (e.g., biliary stone, biliary calculi) required procedures such as laparotomy and open common bile duct exploration, a surgery that was associated with significant mortality, morbidity, and/or prolonged inpatient convalescence. With the advent of ERCP, anatomical obstruction extraction can be accomplished by a gastroenterologist or other qualified practitioners as an outpatient procedure with minimal risks.

The ERCP procedure involves the introduction of a side-viewing endoscope such as a duodenoscope through the mouth of a subject and advancement into the second portion of the duodenum, with the subject positioned semi-prone on an X-ray table for fluoroscopic imaging. The tip of the duodenoscope is aligned with the bile duct and pancreatic duct openings at a shared protuberance called the ampulla or papilla of Vater. The papilla of Vater has an opening called the ampullary orifice surrounded by a sphincter muscle called the sphincter of Oddi. Thereafter, imaging may be performed to provide an assessment of the biliary tract and associated anatomical structures. If a blockage or other anatomical obstruction is detected or found, a device may be inserted through a lumen of the endoscope and/or inserted adjacent or proximate to an endoscope for the purposes of removing the anatomical obstruction from the biliary tract and associated anatomical structures. To address many unmet needs in the state-of-the-art, the present disclosure provides anatomical obstruction extraction devices and associated methods which effectively remove, transport, and/or clear anatomical obstructions from various body lumens and/or cavities of a subject.

Although configurations of the present disclosure may be described with specific reference to medical devices and systems (e.g., endoscopic devices, accessory tools, and/or guidewires inserted through a duodenoscope, near or through a papilla, or the like) for selective access to, aligning with, and/or cannulation of the common bile duct (CBD) or pancreatic duct (PD) during an Endoscopic Retrograde Cholangiopancreatography (ERCP) procedure, such medical devices and systems may be used in a variety of medical procedures which require navigating one or more accessory tools through ductal, luminal, vascular, or body lumen anatomies, including, for example, interventional radiology procedures, balloon angioplasty/angiography procedures, thrombolysis procedures, urological or gynecological procedures, and the like. The disclosed medical devices and systems may be inserted via different access points and approaches, e.g., percutaneously, endoscopically, laparoscopically, or some combination thereof.

In some instances, a medical device may be adapted for removing stones from a body lumen. The medical device may include an elongate shaft, an inner loop that is extendable from the elongate shaft within an inner loop plane, and an outer loop that is extendable from the elongate shaft within an outer loop plane. The inner loop and/or the outer loop may be movable between a capture configuration in which the inner loop plane is transverse (e.g., non-parallel, substantially orthogonal, orthogonal, etc.) to the outer loop plane and a release configuration in which the inner loop plane is at least substantially parallel with the outer loop plane. Substantially orthogonal may be defined as the inner loop plane and the outer loop plane intersecting one another at an angle ranging from 70 degrees to 110 degrees (90 degrees ±20 degrees). Substantially parallel may be defined as an intersecting line that is orthogonal to one of the inner loop plane and the outer loop plane intersecting the other of the inner loop plane and the outer loop plane at an angle ranging from 70 degrees to 110 degrees (90 degrees ±20 degrees).

In some cases, the inner loop may be adapted to be rotatable relative to the outer loop. In some cases, the elongate shaft may be adapted to allow the inner loop to translate relative to the elongate shaft. The elongate shaft may be adapted to reversibly collapse the outer loop. In some cases, the inner loop and the outer loop may each include a nickel-titanium alloy.

In some cases, the medical device may further include a handle that is secured to the elongate shaft. The handle may be adapted to allow a user to translate the inner loop relative to the elongate shaft. In some cases, the handle may be adapted to allow a user to rotate the inner loop relative to the outer loop. In some cases, the elongate shaft may include an outer tubular member, an inner tube that extends within the outer tubular member, and a core wire that extends within the inner tube. In some cases, the inner loop may be connected to the core wire and the outer loop may be connected to the inner tube.

The handle may include a handle body, a knob that is rotatably secured relative to the handle body, and a finger ring that is slidingly secured relative to the handle body. The knob may be operably coupled to the core wire such that rotation of the knob causes rotation of the core wire. The finger knob may be operably coupled to the core wire such that sliding the finger ring relative to the handle body causes translation of the core wire. Alternatively or additionally, the handle may include the handle body, the knob that is rotatably secured relative to the handle body, and a sliding element that is slidingly secured relative to the handle body, where an inner tube extends distally from the handle and the core wire within the inner tube.

In some instances, the medical device may be adapted for removing stones from a body lumen. The medical device may include the elongate shaft have an outer tubular member, an inner tube that extends within the outer tubular member, and a core wire that extends within the inner tube. The inner loop may be secured to the core wire and the outer loop may be secured to the inner tube. The inner loop and/or the outer loop may be movable between the capture configuration and the release configuration.

In some cases, the inner loop may be adapted to be rotatable between the capture configuration and the release configuration. In some cases, withdrawing the core wire proximally relative to the inner tube may cause the inner loop to collapse. In some cases, advancing the outer tubular member distally relative to the inner tube may cause the outer tubular member to collapse the outer loop.

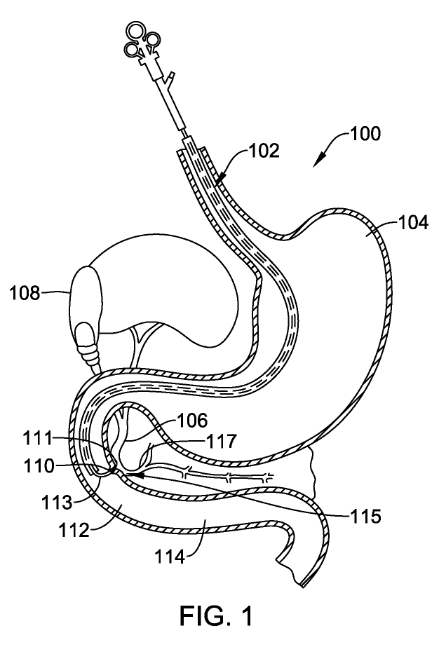

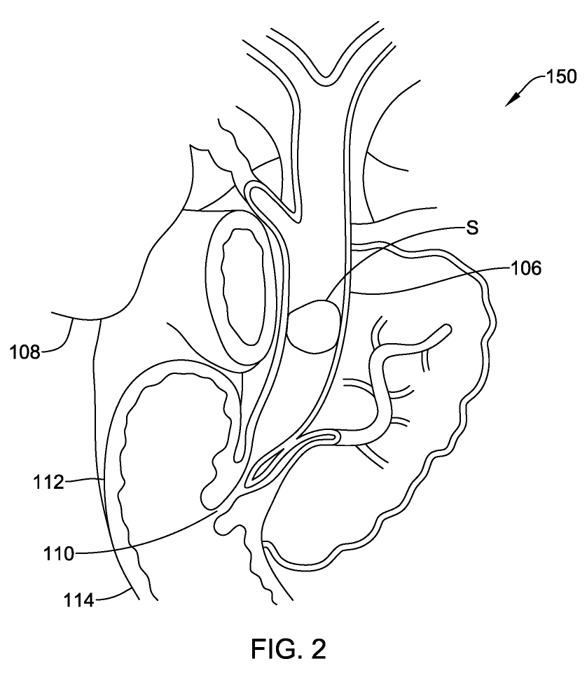

FIG. 1 depicts a schematic view of an example procedural site 100 (such as a biliary and digestive tract) of a subject where an endoscope 102 (e.g., a duodenoscope, etc.) or like introducer bearing an accessory medical device 113 has been inserted into the gastrointestinal tract, through the stomach 104, into the small intestine 114, and into the papilla of Vater 110 by way of the duodenum 112. The biliary duct 106 leads to the gallbladder 108, and forms part of the biliary tract in which anatomical obstructions can form and further accrete into large anatomical obstructions, such as an anatomical obstruction S (e.g., a stone and/or other suitable anatomical structure including, but not limited to, a biliary calculi, mass, osteoid, clot, tissue deformity, tissue anomaly, luminal deformity, luminal anomaly, etc.) shown in FIG. 2.

More specifically, FIG. 1 depicts a schematic view of an illustrative selective cannulation during an ERCP procedure, which includes a guidewire 111 and/or the accessory medical device 113 (e.g., an endoscopic accessory device, such as a sphincterotome, an obstruction removal device, a stone removable device, and/or other suitable accessory medical device) being passed towards, against, and/or through a body lumen such as the papilla of Vater 110 (e.g., ampullary entry) near the duodenum 112 to access the Sphincter of Oddi Complex 115. During the cannulation procedure, a distal portion of the endoscope 102 may be positioned within the duodenum 112. The guidewire 111 and the accessory medical device 113 may be advanced through a working channel (e.g., a lumen) of the endoscope 102 towards the papilla of Vater 110. Additionally, the guidewire 111 and/or the accessory medical device 113 may be advanced against and/or through the papilla of Vater 110 to one of the biliary duct 106 and the pancreatic duct 117.

FIG. 2 depicts a schematic magnified view of a biliary tract of a subject (e.g., a patient). As depicted, an anatomical obstruction S may be present within the biliary duct 106 and require treatment by the medical devices disclosed herein. As shown in FIG. 2, the papilla of Vater 110 provides an access point to the biliary duct 106 from the duodenum 112 of the small intestine 114 such that the medical devices disclosed herein may achieve access to the anatomical obstruction S and remove the anatomical obstruction S from the biliary duct 106 as will be further described herein.

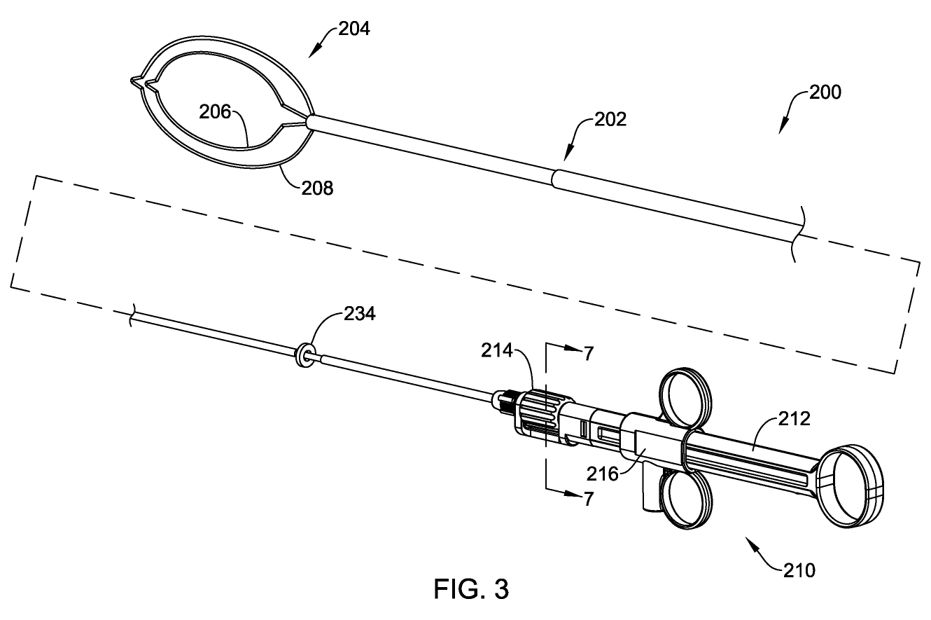

FIG. 3 is a schematic view of an example medical device 200. The example medical device 200 may be an example of the accessory medical device 113 shown in FIGS. 1 and 2, and may be used in combination with the endoscope 102. The medical device 200 may be considered as being adapted for removing obstructions such as stones S from a body lumen such as the biliary duct 106. Additionally or alternatively, the medical device 200 may be considered as being adapted for removing any of a variety of different obstructions from any of a variety of different body lumens.

The medical device 200 may include an elongate shaft 202. As shown, the elongate shaft 202 may include a change in scale. This is merely an artifact of the drawing, as the individual components making up the elongate shaft 202 may be consistent or inconsistent in dimension from one end to another. A basket 204 may extend distally from the elongate shaft 202. In some cases, as shown, the basket 204 may include an inner loop 206 and an outer loop 208, each of which may extend distally from the elongate shaft 202. In some cases, as will be discussed, the elongate shaft 202 may be adapted to allow the inner loop 206 to translate relative to the elongate shaft 202. In some cases, as will be discussed, the elongate shaft 202 may be adapted to reversibly collapse the outer loop 208.

The inner loop 206 and the outer loop 208 may be formed from any suitable materials. Example suitable materials include, but are not limited to, metals, polymers, shape memory materials, stainless steel, nickel-titanium alloys (e.g., NITINOL). In some examples, the inner loop 206 and/or the outer loop 208 may be formed of stainless steel. In some cases, the inner loop 206 and/or the outer loop 208 may be formed of a shape memory material, such as a nickel-titanium alloy and/or other suitable shape memory material.

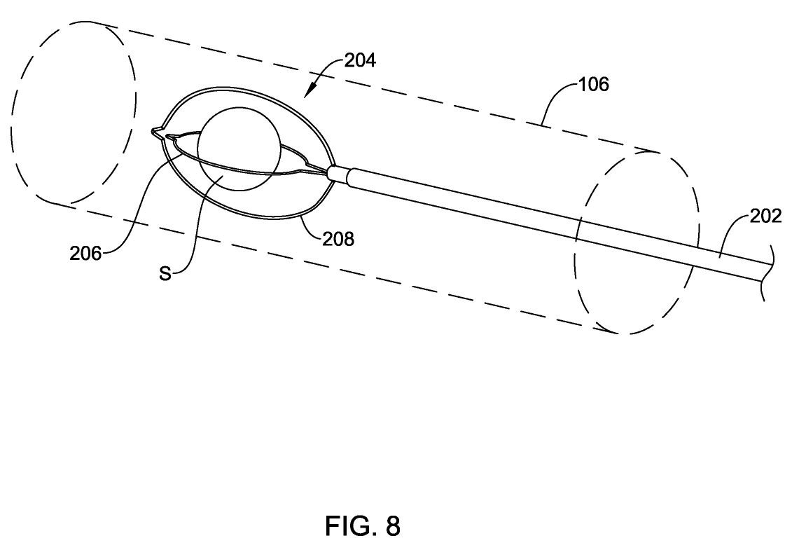

In some cases, the inner loop 206 and/or the outer loop 208 may be movable between a capture configuration (e.g., as seen in FIG. 8) and a release configuration (e.g., as seen in FIG. 3). In some cases, the inner loop 206 is rotatable relative to the outer loop 208 in order to switch between the capture configuration and the release configuration. In some cases, the inner loop 206 and the outer loop 208 may be at least transverse to each other in a capture configuration that is intended to hold an obstruction such as a stone S within the basket 204. In some cases, the inner loop 206 and the outer loop 208 may be at least substantially parallel to each other in a release configuration that is intended to allow the obstruction such as a stone S to be released from the basket 204. In some cases, having a stone S within the basket 204 creates a larger assembly than can easily be withdrawn from the body lumen such as the biliary duct 106. In some cases, it may be necessary to release an obstruction after capturing a stone and prior to getting the assembly stuck in the body lumen. Releasing of the obstruction may be accomplished by adjusting the basket 204 from the capture configuration to the release configuration.

In the capture configuration (as illustrated in FIG. 8), the inner loop 206 and the outer loop 208 may be considered as being at least transverse (e.g., non-parallel, substantially orthogonal, etc.) to each other. Substantially orthogonal may be defined as the inner loop 206 and the outer loop 208 intersecting one another at an angle ranging from 70 degrees to 110 degrees (90 degrees ±20 degrees) and/or other suitable angles at which the obstruction is maintained within the basket 204. In the release configuration (as seen in FIG. 3), the inner loop 206 and the outer loop 208 may be considered as being at least substantially parallel to each other. Substantially parallel may be defined as an intersecting line that is orthogonal to one of the inner loop 206 and the outer loop 208 intersecting the other of the inner loop 206 and the outer loop 208 at an angle ranging from 70 degrees to 110 degrees (90 degrees ±20 degrees) and/or other suitable angles at which the obstruction is released from the basket 204.

In some cases, the inner loop 206 may be considered as defining an inner loop plane in which the inner loop 206 lies and the outer loop 208 may be considered as defining an outer loop plane in which the outer loop 208 lies. In the capture configuration, the inner loop plane may be considered as being transverse (e.g., non-parallel, substantially orthogonal, etc.) to the outer loop plane. In the release configuration (as seen in FIG. 3), the inner loop plane and the outer loop plane may be considered as being at least substantially parallel to each other.

The medical device 200 may include a handle 210 that is secured to the elongate shaft 202. In some cases, the handle 210 may be adapted to allow a user to translate the inner loop 206 relative to the elongate shaft 202. The inner loop 206 may be translatable between a position in which the inner loop 206 extends distally out of the elongate shaft 202 and a position (not shown) in which the inner loop 206 has been withdrawn into the elongate shaft 202. The handle 210 may be adapted to allow a user to rotate the inner loop 206 relative to the outer loop 208 in order to move the basket 204 between its capture configuration and its release configuration, for example. In some cases, the handle 210 may include a handle body 212, a knob 214 that is rotatably secured relative to the handle body 212, and a finger ring 216 that is slidingly secured relative to the handle body 212.

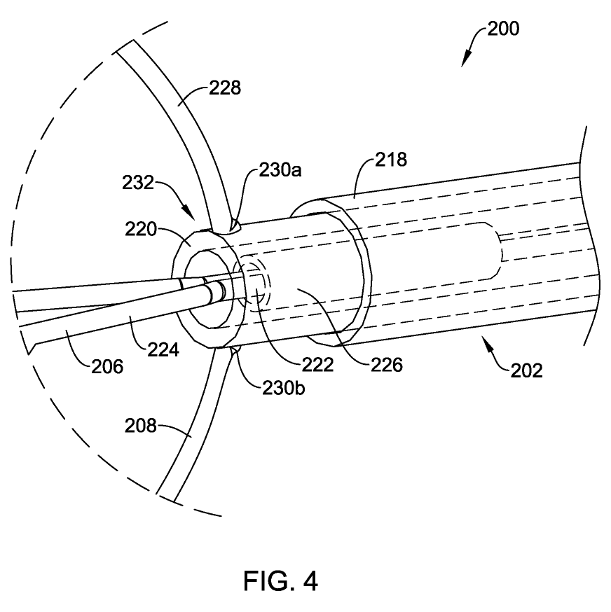

FIG. 4 is an enlarged view of a portion of the medical device 200, showing in greater detail how the inner loop 206 and the outer loop 208 are coupled to the elongate shaft 202. In some cases, the elongate shaft 202 includes an outer tubular member 218. In some cases, the elongate shaft 202 includes an inner tube 220 that extends within the outer tubular member 218. In some cases, the outer loop 208 may be secured to the inner tube 220. The outer loop 208 may be collapsed down into a delivery configuration by advancing the outer tubular member 218 distally over the outer loop 208. In some cases, the elongate shaft 202 may include a core wire 222 that extends within the inner tube 220. In some cases, the inner loop 206 is secured to the core wire 222.

In some cases, the core wire 222 may extend from the inner loop 206 to the handle 210. The core wire 222 passes through the knob 214 and is secured to the finger ring 216. Rotating the knob 214 causes the core wire 222 to rotate, thus rotating the inner loop 206. In some cases, rotating the knob 214 may cause the inner loop 206 to rotate between the capture configuration and the release configuration. Translating the finger ring 216 relative to the handle body 212 may cause the core wire 222 to translate, thus translating the inner loop 206. In some cases, the inner loop 206 may be expanded by translating the finger ring 216 distally relative to the handle body 212, which allows the inner loop 206 to exit the inner tube 220. In some cases, the inner loop 206 may be collapsed by translating the finger ring 216 proximally relative to the handle body 212, which causes the inner loop 206 to be pulled into the inner tube 220. Pulling the inner loop 206 into the inner tube 220 may cause the inner loop 206 to collapse down. It will be appreciated that the knob 214 may rotatably engage the core wire 222 while allowing the core wire 222 to translate relative to the knob 214 as the finger ring 216 is translated distally and proximally relative to the handle body 212.

In some cases, the inner loop 206 may be formed from a metal wire 224 that is heat treated to have a “remembered” shape as shown for example in FIG. 3. In some examples, the inner loop 206 may include one or more other materials on or in the metal wire 224, as a mixture, an alloy, a core, a coating, etc. The “remembered” shape represents a shape into which the inner loop 206 is biased, and will return to absent any constricting forces holding the inner loop 206 in a constricted shape, such as the inner loop 206 being constrained within the inner tube 220. In some cases, the metal wire 224 is doubled over on itself to form the inner loop 206, and the two ends of the metal wire 224 may terminate within a crimp element 226 that is disposed within the inner tube 220. In some cases, the core wire 222 may extend proximally from the crimp element 226 in order to functionally couple the inner loop 206 with the handle 210. In some cases, one or both ends of the metal wire 224 may extend proximally to the handle 210, effectively forming the core wire 222. The metal wire 224 may be formed of a stainless steel, or a nitinol material, for example. In some cases, the metal wire 224 may have, as shown, a circular cross-sectional profile. In some cases, the metal wire 224 may have an ovoid cross-sectional profile or even a polygonal cross-sectional profile.

In some cases, the outer loop 208 may be formed from a metal wire 228 that heat treated to have a “remembered” shape as shown for example in FIG. 3. In some examples, the outer loop 208 may include one or more other materials on or in the metal wire 228, as a mixture, an alloy, a core, a coating, etc. The “remembered” shape represents a shape into which the outer loop 208 is biased, and will return to absent any constricting forces holding the outer loop 208 in a constricted shape, such as the outer loop 208 being constrained within the outer tubular member 218. In some cases, the metal wire 228 may be doubled over on itself to form the outer loop 208. The metal wire 228 may include a first terminal end 230a and a second terminal end 230b, each of which are welded or otherwise secured to a distal region 232 of the inner tube 220. The metal wire 228 may be formed of a stainless steel, or a nitinol material, for example. In some cases, the metal wire 228 may have, as shown, a circular cross-sectional profile. In some cases, the metal wire 228 may have an ovoid cross-sectional profile or even a polygonal cross-sectional profile. In some cases, the inner tube 220 may be formed of a stainless steel or a nitinol material.

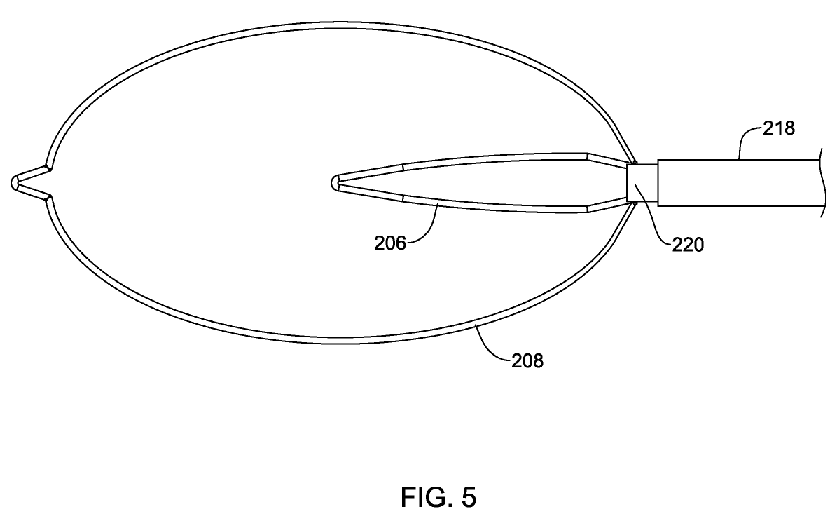

FIG. 5 shows the medical device 200 with the inner loop 206 partially collapsed. This may be achieved by moving the finger ring 216 proximally with respect to the handle body 212, thereby pulling the core wire 222 proximally and thus pulling the inner loop 206 into the inner tube 220. The walls of the inner tube 220 engage the metal wire 224 and cause the inner loop 206 to collapse downward into a configuration that will allow the inner loop 206 to be retracted fully into the inner tube 220. It will be appreciated that the medical device 200 may be advanced through the anatomy to reach a desired treatment site such as the biliary duct 106 with the inner loop 206 fully retracted into the inner tube 220.

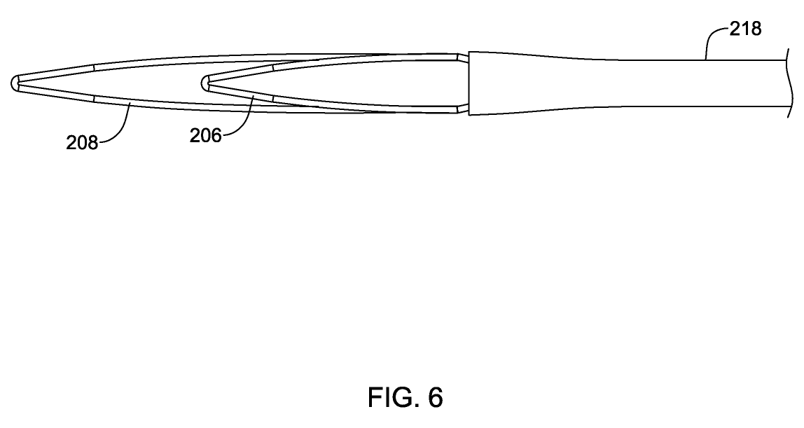

FIG. 6 is similar to FIG. 5, but also shows the outer loop 208 partially collapsed. This may be achieved by advancing the outer tubular member 218 distally (e.g., relative to the inner tube 220) over the outer loop 208 proximally withdrawing the inner tube 220 relative to the outer tubular member 218. In some cases, and with brief reference to FIG. 3, the outer tubular member 218 may include a flared proximal end 234 that may be pulled proximally in order to pull the outer tubular member 218 proximally and may be pushed distally in order to push the outer tubular member 218 distally. As the flared proximal end 234 is pushed distally, the outer tubular member 218 moves distally and begins to collapse the outer loop 208 as the outer tubular member 218 begins to move over the outer loop 208. By continuing to push the outer tubular member 218 distally, the outer loop 208 may be fully collapsed into an interior of the outer tubular member 218. It will be appreciated that the medical device 200 may be advanced through the anatomy to reach a desired treatment site such as the biliary duct 106 with the outer loop 208 fully collapsed within the outer tubular member 218 (and the inner loop 206 fully collapsed within the inner tube 220).

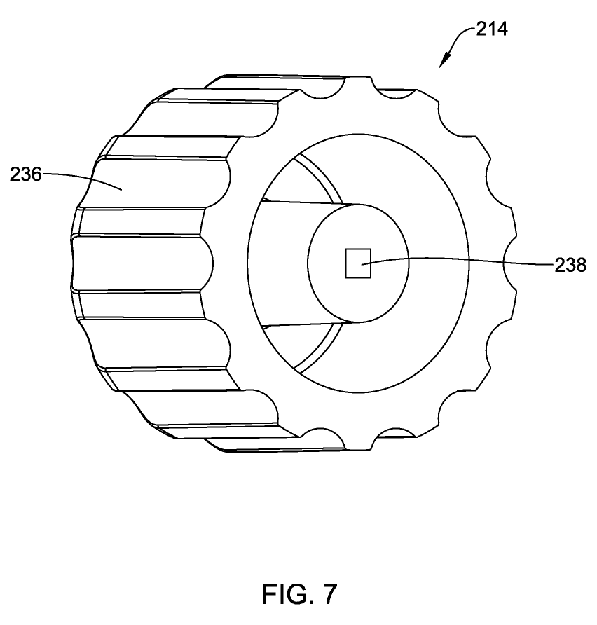

As noted, the knob 214 may rotatably engage the core wire 222 while allowing the core wire 222 to translate relative to the knob 214 as the finger ring 216 is translated distally and proximally relative to the handle body 212. FIG. 7 is a cross-sectional view of an example configuration of the knob 214. The knob 214 may have a knurled outer surface 236 that allows a user to easily rotate the knob 214 relative to the handle body 212. The knob 214 may include a keyed passage 238 that allows the core wire 222 to translate relative to the knob 214 but does not allow the core wire 222 to rotate relative to the knob 214. As shown, the keyed passage 238 may have a square cross-sectional profile. While not illustrated, the core wire 222, or at least a portion of the core wire 222 passing through the knob 214, may have a corresponding square cross-sectional profile. As a result, rotating the knob 214 may cause a similar rotation of the core wire 222. In some cases, rotating the knob 214 about ninety degrees may cause a corresponding rotation of the core wire 222 of about ninety degrees. In some cases, this may result in a corresponding rotation of the inner loop 206 of about ninety degrees or less, depending on the torqueability of the core wire 222. While a square cross-sectional profile is shown for the keyed passage 238 (and for the unseen core wire 222), the keyed passage 238 may have a three-sided cross-sectional profile (triangular), a four-sided rectilinear cross-sectional profile (non-square) or a five or more-sided polygonal cross-sectional profile, as long as the core wire 222, or at least a portion of the core wire 222 passing through the knob 214, may have a corresponding complementary profile.

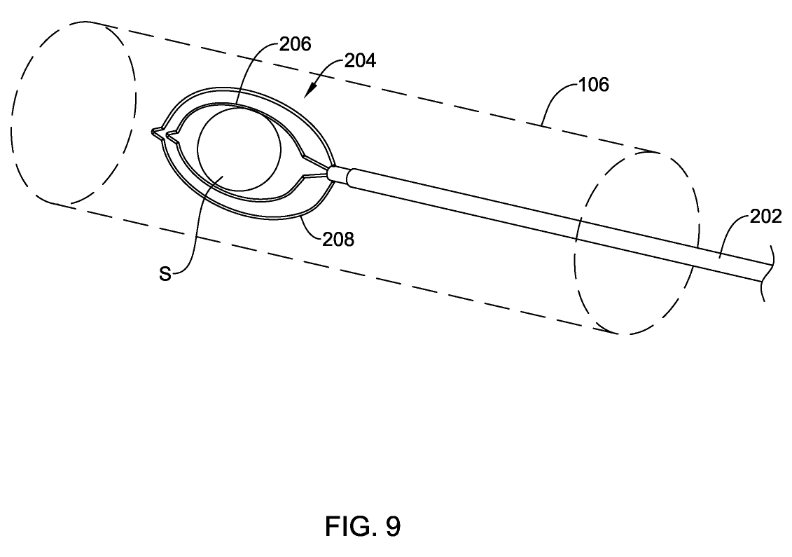

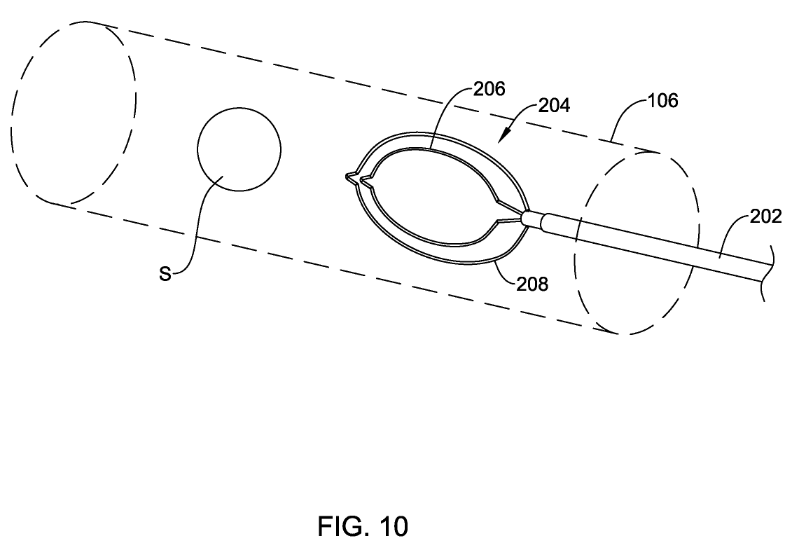

FIGS. 8, 9 and 10 provide an example of how the inner loop 206 may be manipulated relative to the outer loop 208 in order to selectively achieve the capture configuration or the release configuration. In FIG. 8, a stone S is shown captured within the basket 204, within the biliary duct 106. The basket 204 may be formed by the inner loop 206 being at least transverse (e.g., substantially orthogonal, as depicted in FIG. 8) with the outer loop 208. For this example, assume that the stone S is stuck within the basket 204, and the basket 204 may not be withdrawn from the biliary duct 106 with the stone S remaining within the basket 204 due to the basket 204 and stone creating an assembly that is too large to be withdrawn from the biliary duct through the lumen. When this happens, the user is able to rotate the inner loop 206 relative to the outer loop 208 by rotating the knob 214. This causes the inner loop 206 to rotate to a position in which the inner loop 206 is substantially parallel with the outer loop 208, as shown in FIG. 9. The medical device 200 may now be pulled proximally relative to the stone S, as shown in FIG. 10. Once the stone S has been released, the inner loop 206 and the outer loop 208 may each be collapsed and withdrawn into the elongate shaft 202 and the medical device 200 may be repositioned to go after another stone S, or may be withdrawn in favor of a different process for removing the stone S.

The materials that can be used for the various components of the systems presently disclosed may include those commonly associated with medical devices. The devices and/or components thereof may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium- molybdenum alloys (e.g., UNS: R30035 such as MP35-N® and the like), nickel- molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include, but are not limited to, polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some configurations the sheath can be blended with a liquid crystal polymer (LCP). For example, the mixture can contain up to about 6 percent LCP.

In some configurations, a degree of Magnetic Resonance Imaging (MRI) compatibility may be imparted. For example, the devices described herein, or portions thereof, may be made of a material that does not substantially distort the image and create substantial artifacts (i.e., gaps in the image). Certain ferromagnetic materials, for example, may not be suitable because they may create artifacts in an MRI image. The devices described herein, or portions thereof, may also be made from a material that the MRI machine can image. Some materials that exhibit these characteristics include, for example, tungsten, cobalt-chromium-molybdenum alloys (e.g., UNS: R30003 such as ELGILOY®, MRIMRIMRIUNSUNSPHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example configuration being used in other configurations. The invention's scope is, of course, defined in the language in which the appended claims are expressed.

What is claimed is:

1. A medical device adapted for removing stones from a body lumen, the medical device comprising:
an elongate shaft;
an inner loop extendable from the elongate shaft within an inner loop plane;
an outer loop extendable from the elongate shaft within an outer loop plane;
wherein the inner loop and/or the outer loop are movable between a capture configuration in which the inner loop plane is substantially transverse to the outer loop plane and a release configuration in which the inner loop plane is at least substantially parallel with the outer loop plane.

2. The medical device of claim 1, wherein the inner loop is adapted to be rotatable relative to the outer loop.

3. The medical device of claim 1, wherein the elongate shaft is adapted to allow the inner loop to translate relative to the elongate shaft.

4. The medical device of claim 1, wherein the elongate shaft is adapted to reversibly collapse the outer loop.

5. The medical device of claim 1, wherein the inner loop and the outer loop each comprise nitinol.

6. The medical device of claim 1, further comprising a handle secured to the elongate shaft.

7. The medical device of claim 6, wherein the handle is adapted to allow a user to translate the inner loop relative to the elongate shaft.

8. The medical device of claim 6, wherein the handle is adapted to allow a user to rotate the inner loop relative to the outer loop.

9. The medical device of claim 1, wherein the elongate shaft comprises:
an outer tubular member;
an inner tube extending within the outer tubular member; and
a core wire extending within the inner tube.

10. The medical device of claim 9, wherein:
the inner loop is connected to the core wire; and
the outer loop is connected to the inner tube.

11. A medical device adapted for removing stones from a body lumen, the medical device comprising:
an elongate shaft including:
an outer tubular member;
an inner tube extending within the outer tubular member; and
a core wire extending within the inner tube;
an inner loop secured to the core wire; and
an outer loop secured to the inner tube;
wherein the inner loop and/or the outer loop are movable between a capture configuration and a release configuration.

12. The medical device of claim 11, wherein:
in the capture configuration, the inner loop is at least substantially orthogonal to the outer loop; and
in the release configuration, the inner loop is at least substantially coplanar with the outer loop.

13. The medical device of claim 12, wherein the inner loop is adapted to be rotatable between the capture configuration and the release configuration.

14. The medical device of claim 11, wherein withdrawing the core wire proximally relative to the inner tube causes the inner loop to collapse.

15. The medical device of claim 11, wherein advancing the outer tubular member distally relative to the inner tube causes the outer tubular member to collapse the outer loop.

16. The medical device of claim 11, further comprising a handle secured to the elongate shaft, the handle including:
a handle body;
a knob rotatably secured relative to the handle body, the knob operably coupled to the core wire such that rotation of the knob causes rotation of the core wire; and
a finger ring slidingly secured relative to the handle body, the finger knob operably coupled to the core wire such that sliding the finger ring relative to the handle body causes translation of the core wire.

17. A medical device adapted for removing stones from a body lumen, the medical device comprising:
a handle including:
a handle body;
a knob rotatably secured relative to the handle body; and
a sliding element slidingly secured relative to the handle body;
an inner tube extending distally from the handle;
a core wire extending distally from the core wire within the inner tube;
an inner loop extending distally from the core wire; and
an outer loop extending distally from the inner tube;
wherein the inner loop and/or the outer loop are movable between a capture configuration and a release configuration.

18. The medical device of claim 17, wherein:
in the capture configuration, the inner loop is at least substantially orthogonal to the outer loop; and
in the release configuration, the inner loop is at least substantially coplanar with the outer loop.

19. The medical device of claim 17, further comprising an outer tube adapted to slidingly fit over the inner tube.

20. The medical device of claim 19, wherein the outer tube is adapted to be pushed distally in order to close the outer loop.
